(19) United States
(12) Patent Application Publication
(10) Pub. No.: US 2009/0221462 A1
(43) Pub. Date: Sep. 3, 2009

(54) Fragrance compositions

(76) Inventors: Hifzur Rahman Ansari, Old Tappan, NJ (US); Philip William Goulding, Kent (GB); Keith Douglas Perring, Kent (GB)

Correspondence Address: MORGAN LEWIS & BOCKIUS LLP, 1111 PENNSYLVANIA AVENUE NW, WASHINGTON, DC, 20004, US

(21) Appl. No.: 11/921,851

(22) PCT Filed: Jun. 7, 2006
(86) PCT No.: PCT/GB2006/002095

§ 371 Date: Mar. 31, 2009

(30) Foreign Application Priority Data
Jun. 8, 2005 (GB) 0511623.1

(51) Int. Cl. A61K 8/368; A61K 8/36; C11D 3/50

(52) U.S. Cl. 510/102; 512/21; 512/8

(57) ABSTRACT

Fragrance compositions containing volatile organic acids may be used to eliminate amine malodors from an air-space. The acids are characterised by having (i) a pKa at 250C of from 3 to 5, preferably a pKa of from 3.5 to 4.5; (ii) a vapour pressure, at 250C, greater than 0.1 μm Hg, preferably greater than 1 μm Hg, even more preferably between 1 and 100 μm Hg. Preferred acids are cinnamic acid, phenylacetic acid, benzoic acid and 2-pentycyclopropanecarboxylic acid, and combinations thereof, present at levels of at least 0.2% by weight, more preferably 0.5% by weight in the fragrance.

### FIELD OF THE INVENTION

This invention relates to fragrance compositions. For the purposes of this invention a perfume composition is defined as a mixture of perfume ingredients, if desired mixed with or dissolved in a suitable solvent or solvents and/or mixed with a solid substrate. Perfume ingredients are well known to those skilled in the art, and include those mentioned, for example, in S. Arctander, Perfume and Flavor Chemicals (Montclair, N.J., 1969), in S. Arctander, Perfume and Flavor Materials of Natural Origin (Elizabeth, N.J., 1960) and in “Flavor and Fragrance Materials-1991”, Allured Publishing Co. Wheaton, Ill. USA. Perfume ingredients may include natural products such as extracts, essential oils, absolutes, resinoids, resins, concretes etc., and also synthetic basic substances such as hydrocarbons, alcohols, aldehydes, ketones, ethers, acids, esters, acetals, ketals, nitrites, etc., including saturated and unsaturated compounds, aliphatic, aromatic and heterocyclic compounds. The invention is particularly concerned with fragrance compositions that have the ability to combat malodors, particularly airborne amine malodors. The term “combat” is used in relation to malodors in a wide sense, to cover reduction or neutralisation of malodors by any means without any implications as to mechanism.

### BACKGROUND TO THE INVENTION

A large number of malodors surround us, and they take many forms and arise from numerous sources. Examples of particularly pungent malodors include the characteristic odor of ammonia; the fishy odors associated with amines; the classic rotten egg smell of hydrogen sulfide; the fecal odor of skatole; cadaverine has the odor of decaying flesh; and indole or methyl mercaptan emit a skunk or rotten cabbage odor.

There are numerous sources of amine malodors, including land fills, cat litter, chicken coops, water treatment plants and ponds, garbage, dog kennels, rendering plants, food processing plants, wool plants, fish canneries, sewers, paper mills and rest rooms.

Amine malodors can emanate from organic waste products via chemical or microbial degradation and are a public nuisance as well as a health hazard. For example, amine malodors may be formed as the end product in the degradation of urea by the enzyme urease, and the enzyme degradation of uric acid to form urea.

The control of amine odors is a major problem. Many efforts have been made to solve this and related odor problems. The prior art is concerned generally with the treatment of the sources of amine malodors to prevent or minimize the emission of these malodors into the air. For example, U.S. Pat. No. 3,944,408 (Postrihac) refers to the treatment of sewage sedimentation with spent sulfite liquor; U.S. Pat. No. 4,127,383 (Johnston et al) describes the use of a salt of lignosulfonic acid and a foaming agent for treating amine and proteinaceous waste; and U.S. Pat. No. 3,989,498 (Cox) contains the use of glacial acetic acid and amyl alcohol as a deodorant.

In general, the prior art is directed towards the use of materials such as acids to prevent or minimize the emission of amine malodors from solid or liquid substrates.

It is known to use acids to counteract ammonia malodors. Examples include the disclosures in U.S. Pat. No. 3,059,615, U.S. Pat. No. 3,029,783, U.S. Pat. No. 4,306,516 and U.S. Pat. No. 3,892,846. These patents all suggest the use of inorganic or organic acids to treat animal litter to control ammonia formation. These acids are essentially used to neutralize ammonia. As ammonia is a basic material, maintaining a low pH will result in the formation of salts of ammonia, which are generally odorless.

While the use of acids is known to reduce the emission of malodors from solid and/or liquid substrates, the use of selected acids within fragrance compositions to counteract malodors in the gaseous phase has not previously been considered. One additional major disadvantage of the use of acids in fragrance compositions is that they are considered to be harmful to the desired odors of the fragrance compositions.

Other methods of controlling amine malodors include the use of masking materials—these are generally fragrance compositions. However, these materials have a major drawback in that the masking effect is merely a superficial camouflage and is not a true elimination of the malodor.

In addition, the general understanding in the art is that amine malodors may be too intense to be effectively masked by a perfume composition.

### SUMMARY OF THE INVENTION

In one aspect, the present invention provides a fragrance composition, comprising at least 0.2% by weight of at least one volatile organic acid, wherein said volatile organic acid is characterized by having (i) a pKa at 25° C. of from 3 to 5; and (ii) a vapour pressure, at 25° C., greater than 0.1 μm Hg.

The fragrance compositions of the invention can have the ability to combat malodors, particularly amine malodors, especially airborne amine odors, including alkylamines such as trimethylamine and isobutylamine, and heterocyclic amines such as skatole, indole and pyridine. The invention thus provides a malodor combating fragrance composition, particularly an amine malodor combating fragrance composition, comprising a fragrance composition in accordance with the invention.

The term “malodor combating fragrance composition” is used in this specification to mean a composition that produces a discernible reduction in malodor in an environment, particularly a gaseous environment such as the ambient atmosphere, especially in a confined space such as a room space, after exposure of the environment to the composition. The reduction in malodor can conveniently be detected by a trained sensory evaluator, that is a person having skill in physical perfume/odor evaluation who has been screened by an odor sensory test and who has been trained as a sensory evaluator for at least six months.

The term “amine malodor combating fragrance composition” is used particularly to mean a composition that reduces isobutylamine levels by at least 90%, preferably at least 95%, when tested using the headspace test of Example 1, namely a headspace test using 0.5 g of the composition exposed for 30 minutes to 1 ml isobutylamine saturated vapour at 25° C.

In particular, the present invention provides malodor combating fragrance compositions that are effective in combating amine malodors wherein the malodor combating fragrance compositions are volatilized into the atmosphere containing airborne amine malodor(s).

Having regard to the actual status of the known art describing the masking of the prevention of emission of gaseous malodors, the present invention provides fragrance compositions that can be highly effective at combating airborne amine malodors.

Although there are well-known methods and compositions that prevent or minimize the emission of amine malodors and methods that mask amine malodors once they have been emitted into the air, prior to this invention it was not known that such malodors could be combated effectively after they have been emitted into the atmosphere.

The present invention thus provides malodor combating fragrance compositions, particularly for atmosphere applications. The malodor combating compositions comprise at least one volatile organic acid. Combating of amine malodors may occur via traditional sensory interactions (e.g. masking) but may also occur via interaction of malodor and organic acid, within condensed phases such as fluid droplets (eg from aerosols), solid surfaces (eg clothes, carpets, wall-paper, dust motes); and even, under the right conditions, in situ in the atmosphere.

The volatile organic acids used in the present invention have (i) a pKa at 25° C. of from 3 to 5, preferably a pKa of from 3.5 to 4.5; and (ii) a vapour pressure, at 25° C., greater than 0.1 μm Hg, preferably greater than 1 μm Hg, even more preferably between 1 and 100 μm Hg.

The volatile organic acid forms part of a fragrance composition and should have suitable properties for this, both in terms of physical/chemical properties and hedonic properties. The acid should not be irritant, toxic, harmful etc. at the levels of use. In terms of hedonic properties, the acid should not have an adverse effect on the odor properties of the fragrance composition when used at an appropriate level in a complex mixture of fragrance materials. The volatile organic acid may have little or no odor, thus having little or no effect (other than dilution) on the odor properties of the fragrance composition. Preferably, however, the volatile organic acid has useful odor properties, or “odor utility”, so that it may contribute to and possibly enhance the odor properties of the fragrance composition. The term “odor utility” refers to materials that are odiferous, and capable of being incorporated to good effect in a fragrance composition, by which is meant that the odor acceptability of the fragrance is not degraded, i.e. the odor properties are not adversely affected. Suitable volatile organic acids include known perfumery organic acids, e.g. including those listed in the Arctander and Allured reference works mentioned above. Most perfumes comprise hydrophobic constituents, and acids that are freely soluble in perfumes will therefore tend to be somewhat hydrophobic in nature, as exemplified for example by their water solubilities. Organic acids suitable for use in perfumes therefore tend to be have relatively low water solubility, usually less than 3% by weight at 25° C.

Preferred volatile organic acids are selected from the group consisting of cinnamic acid; phenylacetic acid; benzoic acid and 2-pentylcyclopropanecarboxylic acid. pKa values (at 25° C.) are as follows:
cinnamic acid: trans=4.44, cis=3.89
phenylacetic acid: 4.28
benzoic acid: 4.19

Benzoic acid is substantially odorless, and the other acids have odor utility. Cinnamic acid, benzoic acid and phenylacetic acid are all easily available commercially from a number of suppliers. 2-pentycyclopropanecarboxylic acid is available from Quest International under the trade name “PATCHULAC”.

The fragrance compositions preferably comprise an effective amount of volatile organic acid. An “effective amount” is any amount, determinable by those skilled in the art, capable of measurably improving environmental conditions. Preferably, the at least one volatile organic acid comprises at least 0.2% by weight of fragrance composition, more preferably at least 0.5% by weight. The acid is preferably present in an amount of less than about 10% by weight, more preferably less than about 5% by weight of the fragrance composition. Good results have been obtained with acid levels in the range 0.5 to 2% by weight, e.g. about 1% by weight.

Fragrance compositions containing at least 0.2% by weight of volatile organic acids satisfying the criteria of this invention can be expected to counteract amine malodors better than those that do not.

The fragrance compositions may additionally comprise up to 50%, by weight of the volatile organic acid present, of nitrogenous perfumery materials having high basicity such that the pKa of the conjugate acid is at least 4 at 25° C. Examples of such nitrogenous perfumery materials include indole and methyl anthranilate.

The vapour pressure of various materials is available from reference literature such as the CRC Handbook of Chemistry and Physics, various editions; or can be determined by ASTM D5191, ASTM D323, and ASTM D4953.

The terms “fragrance” and “perfume” herein are used synonymously, and the term “fragrance composition” refers to compositions comprising at least 2 odoriferous materials that together are able to provide a pleasing fragrance. Fragrance compositions are preferably in a liquid state at ambient temperature, although solid fragrances are also contemplated. Fragrance compositions may have relatively simple compositions or may comprise complex mixtures of natural and synthetic chemical components, all of which are intended to provide a pleasant odor or fragrance. Some materials in the perfume art having no odor or very faint odor are used as diluents or evaporation control agents. Non-limiting examples of such low odor materials are dipropylene glycol, glycol ethers such as Dowanol DPM™ (dipropylene glycol methyl ether), diethyl phthalate, triethyl citrate, isopropyl myristate, and benzyl benzoate. These materials are not counted in the definition of perfume ingredients nor in the weight percentage of the fragrance compositions of the present invention.

The invention is in part based upon an understanding of the function of the various critical constituents of the fragrance compositions and of the amine malodors. It is believed that malodor counteraction according to this invention is an effect of gas phase and condensed phase interactions between the fragrance composition and the amine malodor and is at least in part a result of acid-base reactivity.

The acid dissociation constant is a measure of the inherent proton acidity of a molecule. Most commonly, it is referenced to dissociation in water, and expressed as ‘pKa’, the negative logarithm to base 10. pKa values are available for most organic acids (eg as in The Handbook of Chemistry and Physics, 59th edition, CRC Press 1978, and also in later editions), but may also be estimated with reasonable accuracy as described in “pKa Prediction for Organic Acids and Bases”, by Perrin, Dempsey and Serjeant, published by Chapman & Hall (1981). Many perfume ingredients are acid-sensitive, particularly those possessing carbon-carbon double bonds, and esters, imines and aldehydes, and it is known that over time such components will degrade in the presence of acids, particularly acids with pKa's less than 4 or less than around 3. The use of acids in perfumes is therefore generally rare, and when used are generally present at low levels. We have found that the acids of the invention when used in most perfumes at the levels specified can deliver malodor combating benefits without excessive deleterious perfume degradation in terms of hedonic properties.

In one embodiment, at least one volatile organic acid having a vapour pressure, at 25° C., greater than 0.1 μm Hg, preferably greater than 1 μm Hg, even more preferably between 1 and 100 μm Hg, is mixed with at least two fragrance materials in a solution to form a fragrance composition such that the fragrance composition comprises more than 0.2% by wt of the volatile organic acid. It is also understood that the fragrance composition may be prepared by mixing fragrance in a solution in any order with the volatile organic acid. Preferably, the volatile organic acid comprises less than about 10% by weight, even more preferably less than 5%, but more than about 0.5% by weight, of the mixed composition.

It will be clear to those experienced in the perfumery art that the use of combinations of volatile organic acids with odor utility will facilitate creative freedom, allowing more routes to meet the target hedonics of the perfume. Particularly preferred combinations comprise any two of the following acids: cinnamic acid, phenylacetic acid and 2-pentycyclopropanecarboxylic acid, particularly mixtures of cinnamic acid and phenylacetic acid.

The present invention finds application in a wide variety of consumer products, including, for example and not as a limitation to the present invention, air conditioning/heating applications, air fresheners, bathroom care, candles, hygiene products, deodorizers/sanitizers, hard surface cleaners, household cleaning products, pet care, room fresheners and wipes. The invention finds particular application in air care products, especially air fresheners, toilet care products and hard surface cleaners.

The invention thus provides a fragrance composition, particularly an air care product, comprising a fragrance composition in accordance with the invention.

Compositions of the present invention may be used in a number of malodorous amine containing environments. For example, and not as a limitation to the present invention, environments such as cat litter, garbage, kitchens, dog kennels, toilet bowls and rest rooms, and rooms exposed to cigarette smoke.

The fragrance compositions of the invention can be particularly effective in combating amine malodors in the atmosphere by exposing the atmosphere to the composition for an extended period of time (e.g. at least 30 minutes). This exposure can be achieved passively e.g. by having a surface, layer or film of the composition in gaseous communication with the surrounding atmosphere, or can be achieved actively by dispensing the composition into the atmosphere such as in the form of a spray, aerosol, mist or vaporous emission.

The fragrance composition is conveniently dispensed in an amount and time effective to provide a vaporous emission that is delivered to an airspace.

The fragrance compositions can be dispensed in combination with an odorless liquid carrier such as mineral oil or water, and can be formulated with a viscosity effective to allow for aerosolization. The compositions of the invention may be used in a variety of consumer products comprising for example surfactants, emulsifiers, solvents, propellants, preservatives and antimicrobials.

Dispensing of the fragrance compositions and consumer products containing them may be by any conventional means, such as from a vessel containing the fragrance composition, optionally with a valve and nozzle mechanism, an aerosol or non-aerosol spray, a gas, a solid or liquid air freshener, a scented cloth, potpourri, incense, light bulb ring, a candle, a plug-in air freshener, and the like.

The fragrance compositions can be dispensed within or confined area, for example, by pumping air containing the fragrance composition through an air vent, spraying the malodor combating fragrance compositions into the air as a mist using an aerosol or non-aerosol spray, and the like.

The invention also includes within its scope use of a composition or product in accordance with the invention for the purpose of combating amine malodors, particularly airborne amine malodors.

The invention also covers a method of reducing or preventing airborne amine malodors, comprising exposing an airspace to a composition or product in accordance with the invention, preferably releasing the composition or product into the airspace e.g. as an aerosol.

Although this invention has been described in its preferred form with a certain degree of particularity with respect to amine malodor combating fragrance compositions, it is understood that the present disclosure of the preferred form has been made only by way of example, and that numerous changes in the details of structures and composition of the product may be resorted to, as will be apparent to those skilled in the art, without departing from the scope of the invention. The disclosures of the cited references are incorporated by reference herein.

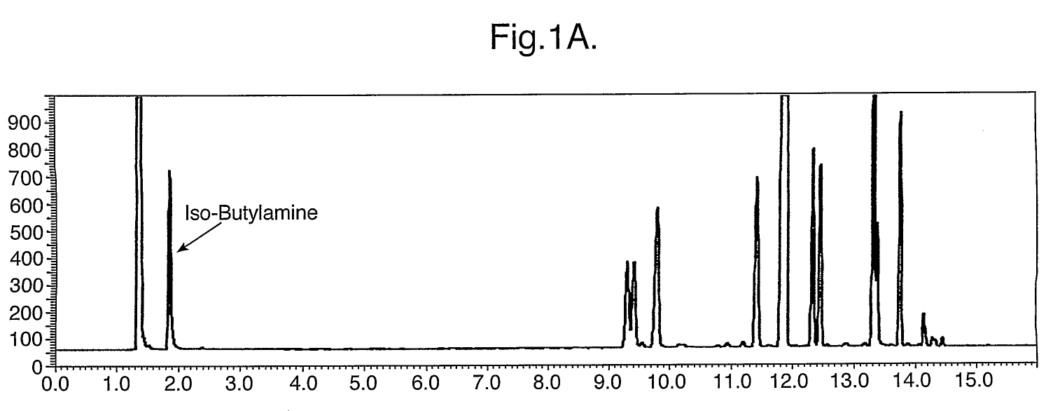

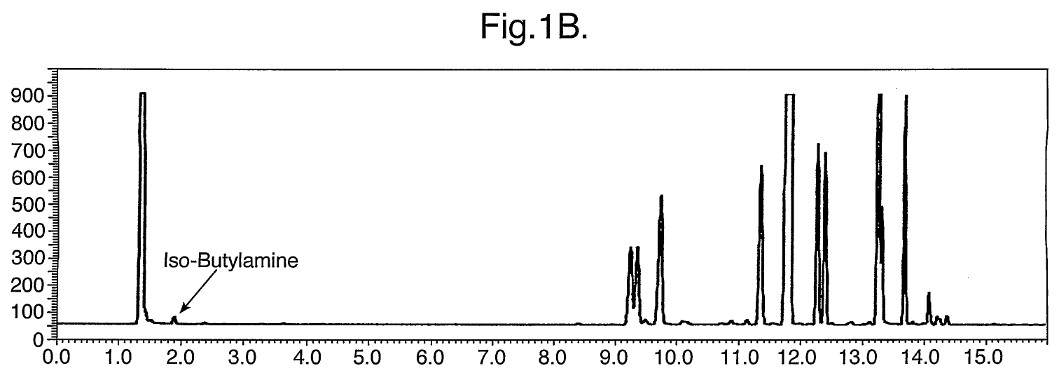

The invention will be further described, by way of illustration, in the following Examples and by reference to the accompanying drawing, in which FIGS. 1A and 1B are a pair of similar headspace analyses.

### EXAMPLES

### Example 1

Mixtures of fragrance ingredients were made up as in Table 1. The essential difference is that mixture 1B contains cinnamic acid whereas mixture 1A does not, and mixture 1A contains slightly more glycol ether solvent (Dowanol DPM) than mixture 1B (expected to have negligible effects on behaviour).

A headspace study was carried out using these two mixtures. An aliquot of each mixture (0.5 g) was added into headspace glass vials (20 ml), and sealed. Neat isobutylamine (an odorous amine found in smoke and other malodor sources) was thermostatted at 25° C. in another vial, and an aliquot (1 ml) of the saturated vapour phase was sampled and injected into each of the vials containing the perfume materials. After 30 minutes, headspace analyses were conducted to determine the headspace concentration of isobutylamine and results are shown in FIG. 1A (for composition 1A) and FIG. 1B (for composition 1B). As shown in FIGS. 1A and 1B, it was found that when cinnamic acid was present (mixture 1B), over 99% of the isobutylamine had been removed from the air in the vial.

TABLE 1
Compositions by weight of compositions 1A and 1B.

w/w %

| Ingredient | 1A | 1B |
|---|---|---|
| Alicate (Q) | 1.5 | 1.5 |
| Allyl heptanoate | 1.5 | 1.5 |
| Benzyl acetate | 5.5 | 5.5 |
| Cinnamic acid | 0 | 1 |
| Dihydromyrcenol | 16 | 16 |
| *Dowanol DPM ™ | 40 | 39 |
| Inonyl acetate | 3.5 | 3.5 |
| IsoAmbois (Q) | 3 | 3 |
| Linalol | 10 | 10 |
| Methyl dihydrojasmonate | 10 | 10 |
| Ortholate (Q) | 6 | 6 |
| Styrallyl acetate | 3 | 3 |
| total | 100 | 100 |

*Dowanol DPM is dipropylene glycol methyl ether.
(Q) indicates a Trade Mark of Quest International.
Alicate is di-isobutyl carbinyl acetate.
IsoAmbois is 1-(2,3,8,8-tetramethyl-1,2,3,4,5,6,7,8-octahydronaphthalen-2-yl) enthanone.
Ortholate is 2-tert.butylcyclohexyl acetate.

This headspace test provides a useful test method for testing the efficacy in reducing amine malodors of compositions. Using this headspace test, a large range of fragrance compositions in accordance with the invention have been tested and all have resulted in a reduction of at least 90% of isobutylamine.

### Example 2

Table 2 below gives details of the formulations of 3 air freshener fragrance compositions in accordance with the invention, perfumes C, D and E, that have good hedonic properties and that are effective in combating airborne amine malodors.

TABLE 2

| | Perfume C | Perfume D | Perfume E |
|---|---|---|---|
| Ingredient | w/w % | w/w % | w/w % |
| Aldehyde C10 | 0.5 | | 0.2 |
| (decanal) | | | |
| Amyl cinnamic | 3 | 2 | 3 |
| aldehyde | | | |
| Benzyl acetate | 7.5 | 10 | 7.5 |
| Bornyl acetate | 30 | 30 | 30 |
| Cinnamic acid | | 1.25 | 1.0 |
| Dihydromyrcenol | 5 | 7.5 | 5 |
| Florida Orange oil | 20 | 14 | 18 |
| Florosa (Q) | 5.5 | 5 | 5 |
| Geraniol | 10 | 6.75 | 7 |
| Patchulac (Q) | 0.5 | 0.5 | |
| Phenylacetic acid | | | 0.3 |
| Phenyl ethyl alcohol, 2- | 14 | 15 | 14 |
| Rossitol (Q) | | 3 | 2.5 |
| Terpinyl acetate | 4 | 5 | 4.5 |
| | 100 | 100 | 100 |

Florosa is p-(2-methyl propyl)-4-hydroxy-4-methyl tetrahydropyran. Patchulac is 2-pentycyclopropanecarboxylic acid. Rossitol is 1-methyl-3-(2-methylpropyl) cyclohexan-1-ol.
Patchulac is 2-pentycyclopropanecarboxylic acid.
Rossitol is 1-methyl-3-(2-methylpropyl) cyclohexan-1-ol.

1. A fragrance composition, comprising at least 0.2% by weight of at least one volatile organic acid, wherein said volatile organic acid is characterized by having (i) a pKa at 25° C. of from 3 to 5; and (ii) a vapour pressure, at 25° C., greater than 0.1 μm Hg.

2. A composition according to claim 1, wherein said acid has a pKa at 25° C. of from 3.5 to 4.5.

3. A composition according to claim 2, wherein said acid has a vapour pressure, at 25° C., of greater than 1 μm Hg.

4. A composition according to claim 3, wherein said acid has a vapour pressure, at 25° C., between 1 and 100 μm Hg.

5. A composition according to claim 1, wherein the at least one volatile organic acid is selected from the group consisting of: cinnamic acid; phenylacetic acid; benzoic acid ad 2-pentycyclopropanecarboxylic acid, and combinations thereof.

6. A composition according to claim 1, wherein the at least one volatile organic acid is present in an amount of at least 0.5% by weight of the composition.

7. A composition according to claim 1, additionally comprising up to 50%, by weight of the volatile organic acid present, of nitrogenous perfumery materials having high basicity such that the pKa of the conjugate acid is at least 4 at 25° C.

8. A composition according to claim 1 for combating malodors, particularly amine malodors.

9. A consumer product comprising a fragrance composition in accordance with claim 1.

10. A product according to claim 9, comprising an air care product.

11. A product according to claim 10, comprising an air freshener, toilet care product or hard surface cleaner.

12. (canceled)

13. A method of reducing or preventing airborne amine malodors, comprising exposing an airspace to a composition or product in accordance with claim 1.

14. A method according to claim 13, wherein the composition or product is released into the airspace.

15. A method according to claim 14, wherein the airspace is exposed to the composition or product for at least 30 minutes.

16. A method for combating airborne amine malodors, comprising exposing said airborne amine malodors to at least one fragrance composition comprising at least one volatile organic acid wherein said volatile organic acid is characterized by having (i) a pKa at 25° C. of from 3 to 5; (ii) a vapour pressure, at 25° C., greater than 0.1 μm Hg.

17. A method according to claim 16, wherein the fragrance composition is released into the atmosphere, e.g. in the form of an aerosol.

18. A method according to claim 17 wherein the fragrance composition comprises at least 0.2% by weight of one or more volatile organic acids selected from cinnamic acid, phenylacetic acid, benzoic acid and 2-pentycyclopropane carboxylic acid and mixtures thereof.

19. A method according to claim 18, wherein the at least one fragrance composition additionally comprises up to 50%, by weight of the volatile organic acid present, of nitrogenous perfumery materials having high basicity such that the pKa of the conjugate acid is at least 4 at 25° C.
